(19) United States
(12) Patent Application Publication
(10) Pub. No.: US 2006/0030578 A1
(43) Pub. Date: Feb. 9, 2006

(54) Pharmaceutically active lipid based formulation of irinotecan

(75) Inventors: Imran Ahmad, Wadsworth, IL (US); Jia-Ai Zhang, Vernon Hills, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NeoPharm, Inc., Lake Forest, IL (US)

(21) Appl. No.: 11/177,194

(22) Filed: Jul. 8, 2005

Related U.S. Application Data
(63) Continuation-in-part of application No. 11/061,044, filed on Feb. 18, 2005.
Application No. 11/061,044 is a continuation of application No. PCT/US03/25880, filed on Aug. 19, 2003.
(60) Provisional application No. 60/404,668, filed on Aug. 20, 2002.

(51) Int. Cl. A61K 31/4745; A61K 9/127

(52) U.S. Cl. 514/283; 424/450

(57) ABSTRACT

Delivering therapeutic amounts of irinotecan remains limited due to its highly water insoluble properties. This invention overcomes this limitation by presenting a novel method of preparing liposomal irinotecan by first inactivating irinotecan prior to liposome formation and then subsequently activating the irinotecan by lowering the pH of the lipid composition to an acidic pH of less than about 3.5, such as between 1.5-3.0 or about 2.

### CROSS-REFERENCE TO RELATED APPLICATIONS

This application is a continuation-in-part of copending U.S. patent application Ser. No. 11/061,044, filed Feb. 18, 2005, which is the national stage application of PCT/US03/25880, filed Aug. 19, 2003, which claims priority to U.S. Provisional Patent Application No. 60/404,668, filed Aug. 20, 2002, the disclosures of which are incorporated herein in their entirety.

### FIELD OF THE INVENTION

This invention pertains to lipid complexes comprising irinotecan and methods for preparing such complexes.

### DESCRIPTION OF THE BACKGROUND

SN38 and irinotecan are exceedingly insoluble in aqueous solutions. Despite this lack of solubility in water, both SN38 and irinotecan have a low affinity for lipid membranes resulting in a tendency to precipitate into the aqueous phase. These solubility characteristics interfere with the use of SN38 and irinotecan as therapeutics. Moreover, the effectiveness of SN38 and irinotecan after repeated administrations can be limited by the development of multi-drug resistance which not only reduces the effectiveness of each compound but also reduces the effectiveness of certain other antineoplastic therapeutics. In addition, the general toxicity of SN38 and irinotecan limits their use therapeutically. Thus, formulations are needed that improve the efficacy of SN38 and irinotecan. Such a formulation should have suitable solubility and toxicity characteristics and will be useful as antiviral agents and in the treatment of certain proliferative diseases such as cancer. The invention provides such compositions and methods. These and other advantages of the present invention, as well as additional inventive features, will be apparent from the description of the invention provided herein.

### SUMMARY OF THE INVENTION

The present invention comprises novel liposomal compositions with high entrapment of irinotecan and the methods of preparing such compositions.

### DETAILED DESCRIPTION OF THE PREFERRED EMBODIMENTS

The inventive composition is a lipid complex with SN38 or irinotecan in which the complex desirably contains cardiolipin. Suitable complexes are characterized by having SN38 or irinotecan bound with a lipophilic compound that imparts solubility characteristics such that stable pharmaceutical preparations can be generated and used. The complexes include, but are not limited to, liposomes, emulsions, and micelles. In the complexes, the SN38 or irinotecan can be bound to the lipid by covalent, hydrophobic, electrostatic, hydrogen, or other bonds and is considered bound even where the SN38 or irinotecan is simply entrapped within the interior of a liposome. The compositions include SN38 or irinotecan complexed with a lipid wherein at least about 40 wt. % or more, such as at least about 50 wt. % or more is complexed with the lipid. More preferably at least about 70 wt. % or more, even more preferably at least about 80 wt. % or more (e.g., at least about 85% or more), and most preferably at least about 90 wt. % or more (such as at least about 95% or more) of the SN38 or irinotecan is complexed with lipid (e.g., at least a portion of the lipid). Where the compositions are liposomal, desirably, at least about 70 wt. % or more, even more preferably at least about 80 wt. % or more (e.g., at least about 85% or more), and most preferably at least about 90 wt. % or more (such as at least about 95% or more) of the SN38 or irinotecan is entrapped or encapsulated with the liposomes.

Desirably, the SN38 or irinotecan lipid complexes contain cardiolipin. Any suitable cardiolipin can be used. For example, cardiolipin can be purified from natural sources or can be chemically synthesized or chemically modified, such as tetramyristylcardiolipin, by such methods as are known in the art.

The complexes generally contain other complexing agents in addition to cardiolipin. Suitable agents include pharmaceutically acceptable synthetic, semi-synthetic (modified natural) or naturally occurring compounds having a hydrophilic region and a hydrophobic region. Such compounds include amphiphilic molecules which can have net positive, negative, or neutral charges or which are devoid of charge. Suitable complexing agents include compounds, such as phospholipids that can be synthetic or derived from natural sources, such as egg or soy. Suitable phospholipids include compounds such as phosphatidylcholine (PC), phosphatidylethanolamine (PE), phosphatidylserine (PS), phosphatidylglycerol (PG), phosphatidic acid (PA), phosphatidylinositol (PI), sphingomyelin (SPM), and the like, alone or in combination. Phosphatidylglycerols such as dimyristoylphosphatidylglycerol, dioleoylphosphatidylglycerol, distearoylphosphatidylglycerol, dipalmitoylphosphatidylglycerol, diarachidonoylphosphatidylglycerol, are preferred, as are mixtures thereof. The phospholipids dimyristoylphosphatidylcholine (DMPC), dimyristoylphosphatidylglycerol (DMPG), dioleoylphosphatidylglycerol (DOPG), distearoylphosphatidyl choline (DSPC), dioleoylphosphatidylcholine (DOPC), dipalmitoylphosphatidylcholine (DPPC), diarachidonoyl phosphatidylcholine (DAPC), egg phosphatidylcholine, soy phosphatidylcholine, or hydrogenated soy phosphatidylcholine (HSPC) can be used, as can mixtures thereof. Other lipids that can be employed include ganglioside GM1 and polymer modified lipids, such as PEG modified lipids.

In addition, the lipid complexes generally include at least one sterol or steroid component such as cholesterol, polyethylene glycol derivatives of cholesterol (PEG-cholesterols), coprostanol, cholestanol, cholestane, or α-tocopherol. They may also contain sterol derivatives such as cholesterol hemisuccinate (CHS), cholesterol sulfate, and the like. Organic acid derivatives of tocopherols, such as α-tocopherol hemisuccinate (THS), can also be used. Suitable complexes can also be formed with glycolipids, or natural or derivatized fatty acids and the like. The preferred complexing agents include cardiolipin (e.g., natural cardiolipin or synthetic cardiolipin), a phosphatidylcholine, cholesterol, and α-tocopherol which are combined to form a liposome.

Any suitable amount of SN38 or irinotecan can be used. Suitable amounts of SN38 or irinotecan are those amounts that can be stably incorporated into the complexes of the present invention. The SN38 or irinotecan should preferably be present in the above mentioned compositions at a concentration of about 0.01 mg/ml to about 20 mg/ml, such as between about 0.1 mg/ml and about 20 mg/ml or between about 0.01 mg/ml and about 5 mg/ml, more preferably about 0.1 to about 4 mg/ml, even more preferably about 0.5 to 3 mg/ml, and most preferably about 0.8 to 2, such as from about 1 or more to about 1.5 mg/ml. Suitable compositions also generally contain from about 1 to about 50 wt. % cardiolipin, or preferably about 2 to about 25 wt. % cardiolipin, or more preferably about 5 wt. % to about 20 wt. % cardiolipin. Such compositions also generally contain about 1 wt. % to about 95 wt. % phosphatidylcholine, or more preferably about 20 wt. % to about 75 wt. % phosphatidylcholine. The preferred compositions also generally contain α-tocopherol in a concentration of about 0.001 wt. % to about 5 wt. %.

The complexing agents can also be considered liposome-forming materials when they are used to generate liposomes by methods such as are known in the art. To generate the desired complexes, they can be dissolved by themselves or with the other lipophilic ingredients, including SN38 or irinotecan, in suitable solvents. Suitable solvents are those which provide sufficient solubility and can be evaporated without leaving a pharmaceutically unacceptable amount of a pharmaceutically unacceptable residue. For example, the cardiolipin can be dissolved in a non-polar or slightly polar solvent such as ethanol, methanol, chloroform, methylene chloride, or acetone. SN38 or irinotecan also can be dissolved in an aqueous, typically alkaline, buffer (e.g., sodium carbonate, sodium bicarbonate, sodium hydroxide, sodium phosphate, sodium acetate, sodium citrate, calcium hydroxide, sodium biphosphate, ammonium acetate, Tris (hydroxy-methyl) aminomethane, sodium benzoate, and the like). The aqueous solution of SN38 or irinotecan can then be added to the lipid film and the resulting mixture vigorously homogenized to produce liposomes, emulsions, and micelles, as desired.

The invention further provides a method for forming a lipid composition comprising SN38 or irinotecan and compounds in equilibrium with SN38 or irinotecan. SN38 and irinotecan can be said to be stable as long as most of the drug retains its chemical structure or a chemical structure that is in equilibrium with its chemical structure. Chemical structures in equilibrium with SN38 or irinotecan specifically include structures that impart greater solubility at high pH but which are converted to SN38 or irinotecan respectively when the pH is lowered.

Generally, the method involves mixing dissolved lipophilic ingredients together and evaporating or lyophilizing the solvent(s) to form a (preferably homogeneous) lipid phase or lipid film. The lipid phase can be formed, for example, in a suitable organic solvent, such as is commonly employed in the art. The lipid phase then is hydrated with a first aqueous solution including SN38 or irinotecan (or compound in equilibrium with SN38 or irinotecan respectively) so as to form a lipid composition including the compound. Thereafter, the pH of the composition is reduced so as to convert some or all of the compounds in equilibrium with SN38 to SN38 or all of the compounds in equilibrium with irinotecan to irinotecan.

Preferably, the lipid phase is a lipid film, which can be generated by known methods. For example, solvent evaporation can be accomplished by any suitable means that preserves the stability of the components. The complexes, including liposomes or micelles, can then be formed by hydrating the lipid phase, such as by adding a suitable solvent to the dry lipid film mixture. Suitable solvents include pharmaceutically acceptable polar solvents. Generally, solvents are aqueous solutions containing pharmaceutically acceptable salts, buffers, or their mixtures. In one method, a lipid film is hydrated with an aqueous solution of SN38 or irinotecan having an alkaline pH. Suitable pHs range from about 7 to about 11, pHs of about 8 to about 10 are more preferred, and pHs of about 9 to about 10 are most preferred. Aqueous solutions having a suitable pH can be prepared from water having an appropriate amount of NaOH dissolved therein. Alternatively, such solutions can be prepared with buffers, such as Tris-HCl, which have pKs within about 1 pH unit of the desired pH. Other suitable buffers include sodium carbonate, sodium bicarbonate, sodium hydroxide, sodium phosphate, ammonium acetate, sodium citrate, sodium hydroxide, calcium hydroxide, sodium biphosphate, sodium phosphate, Tris (hydroxy-methyl) aminomethane, sodium benzoate, and the like.

Liposome complexes can be formed (during the hydration step, for example) by dispersing the lipid in the aqueous solution with vigorous mixing. Any method of mixing can be used provided that the chosen method induces sufficient shearing forces between the lipid film and polar solvent to strongly homogenize the mixture and form the desired complexes. For example, mixing can be by vortexing, magnetic stirring, and/or sonicating. Where multilamellar liposomes are desired, they can be formed simply by vortexing the solution. Where unilamellar liposomes are desired, a sonication or filtration step is included in the process.

Liposomal complexes can be prepared by mixing SN38 or irinotecan, cardiolipin, cholesterol, phosphatidylcholine and α-tocopherol in a suitable solvent to form a homogeneous mixture. The mixture is dried to form a lipid film and hydrated into liposomes by the addition of water or an aqueous solution and mixing. Alternatively, the liposomes can be prepared by dissolving the lipophilic ingredients (with the exception of SN38 or irinotecan) together and evaporating them to form a lipid film. A solution of SN38 or irinotecan is prepared in an aqueous solution at alkaline pH and is then used to hydrate the dry lipid film and form liposomes.

Alternatively, SN38 or irinotecan can be directly dissolved in alkaline aqueous buffer solution, such as previous described. The dissolved SN38 or irinotecan can be added to the liposomes that are prepared by any of the techniques now known or subsequently developed for preparing liposomes. For example, the liposomes can be formed by the conventional technique for preparing multilamellar liposomes (MLVs), that is, by depositing one or more selected lipids on the inside walls of a suitable vessel by dissolving the lipids in chloroform and then evaporating the chloroform, adding the aqueous solution which is to be encapsulated to the vessel, allowing the aqueous solution to hydrate the lipid, and swirling or vortexing the resulting lipid suspension to produce the desired liposomes.

Alternatively, techniques used for producing large unilamellar liposomes (LUVs), such as, reverse-phase evaporation, solvent dilution procedures, infusion procedures, and detergent dilution, can be used to produce the liposomes. A review of these and other methods for producing liposomes can be found in Liposomes, Marc J. Ostro, ed., Marcel Dekker, Inc., New York, 1983, Chapter 1.

In general, any suitable method of forming liposomes can be used so long as it generates liposomal entrapped SN38 or liposomal entrapped irinotecan. Multilamellar vesicles, stable plurilamellar vesicles, and reverse phase evaporation vesicles can be used. As can be appreciated, the present invention is intended to cover SN38 or irinotecan-entrapped liposome compositions, however made.

Suitable liposomes can be neutral, negatively, or positively charged, the charge being a function of the charge of the liposome components and pH of the liposome solution. For example, at neutral pH, positively charged liposomes can be formed from a mixture of phosphatidylcholine, cholesterol and stearyl amine. Negatively charged liposomes can be formed, for example, from phosphatidylcholine, cholesterol, and phosphatidylserine.

After formation of the lipid composition comprising SN38 or irinotecan or compounds in equilibrium with SN38 or irinotecan, the pH of the composition is reduced so as to convert some or all of the compounds in equilibrium with SN38 to SN38 or some or all of the compounds in equilibrium with irinotecan into irinotecan. Desirably, the pH of the composition is less than about 3.5 (e.g., a pH of from about 1 and 3.5, such as between about 1.5 and about 3), and preferably the pH is about 2.0. The pH can be reduced, in accordance with the inventive method, directly after the hydration stage, e.g., by adding an acidic buffer (such as those described herein), or after a step of dehydration (or drying), storage (if desired), and re-hydration (also termed “resuspension” or “reconstitution”), as described herein. Alternatively, the pH can be reduced during the re-hydration of a dried or lyophilized preparation, for example, where an acidic buffer is employed to reconstitute dried liposomes containing SN38 or irinotecan.

Targeting agents can be bound to the complexes such that the complexes can be targeted to particular tissues or organs. The agents can be bound through covalent, electrostatic, or hydrophobic bonds with the complexes. Suitable targeting agents include carbohydrates and proteins (e.g., antibodies, antibody fragments, peptides, peptide hormones, receptor ligands, and mixtures thereof) or other agents as are known to target desired tissues or organs. For example, U.S. Pat. No. 6,056,973, which is herein incorporated by reference, discloses a number of targeting agents and target cells. (See col. 11, ln. 1-41). Methods of preparing suitable conjugates are also disclosed. (See Col. 11, ln. 55-col. 14, ln. 20).

The SN38 or irinotecan complexes can be filtered through suitable filters to control their size distribution. Suitable filters include those that can be used to obtain the desired size range of liposomes from a filtrate. Accordingly, the liposomes produced are preferably treated to reduce their size and to produce a homogeneous population. This may be accomplished by conventional techniques such as extrusion through a filter, preferably of 100 to 800 nm pore size, the filter being either the straight path or tortuous path type. The filter preferably has a pore size of about 5 microns or less, and more preferably about 1 micron or less, such as about 500 nm or less, or even about 200 nm or less or 100 nm or less. Other methods of size reducing the liposomes to a homogenous size distribution are ultrasonic exposure, the French press technique, hydrodynamic shearing, homogenization using, for example, a homogenizer or microfluidization techniques. Alternatively, filtration can occur after formulation in liquid excipients or diluents, as hereinafter described.

Thus, for example, the liposomes can have a diameter (e.g., average mean diameter) of about 5 microns or less, and more preferably, about 1 micron or less, such as about 500 nm or less, or even about 200 nm or less or 100 nm or less. It is preferred that the liposomes used in the present invention have an average mean diameter from about 20 nm to about 1000 nm and preferably from about 100 nm to about 800 nm or from about 100 nm to about 400 nm. An average mean diameter of about 160 nm is particularly preferred.

To improve shelf-life, the present invention provides SN38 or irinotecan liposome preparations which can be stored for extended periods of time without substantial leakage from the liposomes of internally encapsulated materials. The present invention provides SN38 or irinotecan liposome preparations which can be dried or dehydrated to form a dried lipid composition, stored for extended periods of time while dehydrated, and then rehydrated when and where they are to be used, without losing a substantial portion of loaded SN38 or loaded irinotecan during the dehydration, storage and rehydration processes. The drying or dehydration can be achieved either after or before the pH of the composition is reduced.

The liposomes are preferably dried or dehydrated to form a dried lipid composition using standard freeze-drying equipment or equivalent apparatus, that is, they are preferably dehydrated under reduced pressure. If desired, the liposomes and their surrounding medium can be frozen in liquid nitrogen before being dehydrated. Alternatively, the liposomes can also be dehydrated without prior freezing, by simply being placed under reduced pressure.

To achieve these and other objects, the invention, in accordance with one of its aspects, provides SN38 or irinotecan liposome preparations that have been dehydrated in the presence of one or more protective sugars. In certain preferred embodiments of the invention, the liposomes are dehydrated with the one or more sugars being present at both the inside and outside surfaces of the liposome membranes. The sugars are selected from the group consisting of trehalose, maltose, lactose, sucrose, glucose, and dextran, with the most preferred sugars from a performance point of view being trehalose and sucrose. In general, disaccharide sugars have been found to work better than monosaccharide sugars, with the disaccharide sugars trehalose and sucrose being most effective. Other more complicated sugars can also be used. For example, aminoglycosides, including streptomycin and dihydrostreptomycin, have been found to protect liposomes during dehydration. The dehydration is accomplished under vacuum and can take place either with or without prior freezing of the liposome preparation.

Dried, dehydrated, or lyophilized SN38 or irinotecan complex liposomes can be resuspended (i.e., reconstituted) into a suitable solution (typically an aqueous solution) by gentle swirling of the solution. The rehydration can be performed at room temperature or at other temperatures appropriate to the composition of the liposomes and their internal contents. When desired, liposomes can be dried such as by evaporation or lyophilization and the liposomes resuspended (i.e., reconstituted) in any desirable polar solvent. Where liposomes are formed as described herein by hydrating lipid films with alkaline, aqueous solvents containing SN38 or irinotecan, it is desirable to use a low pH buffer, such as those described herein, to resuspend (reconstitute) the dehydrated or lyophilized liposomes. Suitable solvents for resuspending (reconstituting) the liposomes include, for example, a buffered solution (typically an aqueous solution) having a pH of less than about 3.5 (e.g., a pH of from about 1 and 3.5, such as between about 1.5 and about 3), and preferably having a pH of about 2.0 (e.g., a lactate buffered solution having a pH of about 2.0). In such embodiments, the resuspension of the dehydrated lipid composition can effect the reduction of pH of the composition.

When the dehydrated or lyophilized liposomes are to be used, rehydration (or reconstitution) can be accomplished by adding an SN38 or irinotecan activating agent to close the lactone ring of SN38 or irinotecan. In this sense, the SN38 or irinotecan is released as active (pharmaceutically active) SN38 or irinotecan, respectively. Accordingly, the invention provides a method of making SN38 or irinotecan complexes comprising formulating dehydrated or lyophilized complexes containing liposomes and SN38, irinotecan or compounds in equilibrium with SN38 or irinotecan respectively, dissolving or resuspending the dehydrated or lyophilized complexes in an aqueous solution, and contacting the liposomes with an activating agent such that SN38 or irinotecan becomes active. The activating agent can be any acidic aqueous buffer, e.g., sodium citrate, citric acid, sodium acetate, acetic acid, ascorbic acid, sodium lactate, lactic acid, sodium tartrate, tartartic acid, sodium succinate, succinic acid, aspartic acid, hydrochloric acid, meleic acid, sodium carbonate, sodium sulfate, sulfuric acid, preferably, sodium lactate, sodium acetate, and the like. In some embodiments, it can be desirable to employ a solubilizing agent to increase the solubility of SN38 or irinotecan during formulation, such as an alkaline buffer, examples of which are discussed herein. Also, it can be desirable for one or more pharmaceutically acceptable excipients to be employed to increase the shelf-life of the composition. Suitable excipients for enhancing shelf life include, for example, protective sugars, as disclosed herein.

The inventive liposomal compositions desirably are stable for at least about 24 hours, and more preferably, they are stable for at least about 48 hours. Most preferably, the liposomal compositions containing SN38, irinotecan, or other lipid complexes of the present invention, are stable for at least about 72 hours. Stability can be assessed either over the time post-formulation or over the period post-reconstitution following drying or lyophilization. In this context, the stability of a liposomal composition of the present invention over time can be assessed, for example, by assaying the change in mean particle size over a 24, 48, or 72 hour period. Typically, stability is assessed after maintaining the composition at room temperature (e.g., about 25° C.) for the desired period of time, but other suitable temperatures can be employed. Desirably, when measured at 25° C., the mean particle size of the composition after 24, 48, or 72 hours post-formulation or post-reconstitution varies (e.g. is increased or decreased) by less than about 25% (more preferably, the size varies by less than about 20% or 15%, and most preferably by less than about 10% or less than about 5%) of that when the composition is initially formulated or reconstituted. Stability alternatively can be assessed by measuring the pH of the composition over the desired time frame. Desirably, the pH of the composition after 24, 48, or 72 hours post-formulation or post-reconstitution varies (e.g., either is increased or decreased) by at most about 0.5 pH units, and more preferably by at most about 0.4 pH units, from the pH of the composition when the composition is initially formulated or reconstituted. More, preferably, the pH of the composition after 24, 48, or 72 hours post-formulation or post-reconstitution varies by at most about 0.3 pH units from the pH of the composition when initially formulated or reconstituted, and even more preferably by at most about 0.2 pH units from the pH of the composition when initially formulated or reconstituted. Most preferably, the pH of the composition after 24, 48, or 72 hours post-formulation or post-reconstitution varies by at most about 0.1 pH unit from the pH of the composition when initially formulated or reconstituted. Another measurement of stability is the entrapment efficiency of SN38 or irinotecan within the composition, especially a liposomal composition. Desirably, the entrapment efficiency of SN38 or irinotecan within the composition after 24, 48, or 72 hours post-formulation or post-reconstitution is at least about 80% (more preferably at least about 85%, and most preferably at least about 90% or at least about 95%) of that when the composition is initially formulated or reconstituted. Most preferably, the entrapment efficiency of SN38 or irinotecan within the composition measured 24, 48 or 72 hours post formulation or reconstitution does not appreciably change from that measured when the composition is first formulated or reconstituted.

The invention includes pharmaceutical preparations, which, in addition to non-toxic, inert pharmaceutically suitable excipients, contain the SN38 or irinotecan complex and methods for preparing such compositions. By non-toxic, inert pharmaceutically suitable excipients there are to be understood solid, semi-solid or liquid diluents, fillers and formulation auxiliaries of all kinds.

The invention also includes pharmaceutical preparations in dosage units. This means that the preparations are in the form of individual parts, for example capsules, pills, suppositories and ampoules, of which the content of the SN38 or irinotecan complex corresponds to a fraction or a multiple of an individual dose. The dosage units can contain, for example, 1, 2, 3 or 4 individual doses or ½, ⅓ or ¼ of an individual dose. An individual dose preferably contains the amount of SN38 or irinotecan which is given in one administration and which usually corresponds to a whole, a half, a third, or a quarter of a daily dose.

Tablets, dragees, capsules, pills, granules, suppositories, solutions, suspensions and emulsions, pastes, ointments, gels, creams, lotions, powders and sprays can be suitable pharmaceutical preparations.

For the oral mode of administration, the SN38 or irinotecan complex can be used in the form of tablets, capsules, losenges, powders, syrups, aqueous solutions, suspensions, and the like. Carriers such as lactose, sodium citrate, and salts of phosphoric acid can be used to prepare tablets. Further, disintegrants such as starch, and lubricating agents, such as magnesium stearate, sodium lauryl sulfate and talc can be included. Diluents such as lactose and high molecular weight polyethylene glycols can be used in the preparation of dosages in capsule form. The active ingredient can be combined with emulsifying and suspending agents to generate aqueous suspensions for oral use. Flavoring agents such as sweeteners can be added, as desired.

For topical administration and suppositories drug complexes can be provided in the form of such gels, oils, and emulsions as are known by the addition of suitable water-soluble or water-insoluble excipients, for example polyethylene glycols, certain fats, and esters or mixtures of these substances. Suitable excipients are those in which the drug complexes are sufficiently stable to allow for therapeutic use.

The abovementioned pharmaceutical compositions are prepared for administration in the usual manner according to known methods, for example by mixing the complexed SN38 or irinotecan with suitable excipient(s).

A significant advantage of cardiolipin-containing compositions is that they provide a method of modulating multidrug resistance in cancer cells which are subjected to SN38 or irinotecan. In particular, the present compositions reduce the tendency of cancer cells subjected to chemotherapy with SN38 or irinotecan to develop resistance thereto, and reduces the tendency of cancer cells to develop resistance to other therapeutic agents, such as taxol or doxorubicin. Thus, other agents (e.g., secondary therapeutic agents) other than the SN38 or irinotecan complexes can be advantageously employed with the present treatment in combination with the SN38 or irinotecan complexes. Suitable adjunctive secondary therapeutic agents include, for example, antineoplastic agents (such as cisplatin, taxol, doxorubicin, vinca alkaloids, and temozolomide), antifungal agents, antibiotic agents, antiviral agents, antimetabolites, imunomodelators, and other secondary active agents. Preferred secondary agents include, for example, Gonadotropin release hormone, antiestrogens, antiandrogens, cisplatin, carboplatin, oxaliplatin, antisense oligonucleotides, paclitaxel, docetexl, vinca alkaloids, such as vincristin, vinblastine, vindestine and vinorelbine, doxorubincine, daunorubicin, epirubicin, mitoxantrone, cytarabine, temozolomide, leuprolide, cyclophosphamide, etoposide, and Tamoxifen, among other secondary agents.

Having described the present invention, reference will now be made to certain examples which are provided solely for purposes of illustration and which are not intended to be limiting.

### EXAMPLE 1

Irinotecan can be dissolved in chloroform containing cardiolipin. To this mixture, phosphatidylcholine dissolved in hexane and cholesterol in chloroform can be added. The mixture can be stirred gently and the solvents can be evaporated under vacuum to form a thin dry film of lipid and drug. Liposomes can then be formed by adding saline solution and aggressively mixing the components by vortexing. The flasks can then be vortexed to provide multilamellar liposomes and optionally sonicated in a sonicator to provide small unilamellar liposomes. The efficiency of encapsulation can be determined by dialyzing an aliquot of the subject liposomes overnight in a suitable aqueous solvent or centrifuging an aliquot of the subject liposomes. Thereafter the liposome fraction is dissolved in methanol and analyzed by standard methods using high pressure liquid chromatography (HPLC), such as reverse phase HPLC.

### EXAMPLE 2

A lipid film is prepared by adding D-α-tocopherol acid succinate to about t-butyl alcohol which is warmed. The solution is mixed until the tocopherol is dissolved. Tetramyristoyl cardiolipin is added to the solution and the solution is mixed for about 5 minutes. Cholesterol is added to the solution and the solution is mixed for about 5 more minutes then egg phosphatidyl choline is added and mixed for another 5 min. The resulting lipid solution is lyophilized to generate a lipid film.

To prepare liposomal irinotecan, irinotecan is prepared by dissolving the drug in an aqueous alkaline solution having a pH of between 8 and 10. This solution is added to a vial containing the lipid film. The vial is swirled gently, allowed to hydrate at room temperature, vortexed vigorously, and sonicated for 10 min in a bath-type sonicator at maximum intensity. The pH of the liposome solution is reduced to an acidic pH of 2.7.

### EXAMPLE 3

Lipids, DOPC, cholesterol and cardiolipin at the appropriate ratios and tocopheryl acid succinate were dissolved in dichloromethane and subsequently dried under vacuum. The dried lipid film was rehydrated in the irinotecan solution in 10% sucrose in 0.1N NaOH (pH>9). The lipid dispersion was extruded under nitrogen through 0.2 μM and 0.1 μM polycarbonate filters and then lyophilized to yield the LE-irinotecan cake. The lyophilized cake was hydrated with 10 mM lactate buffer (pH 2.0) in order to convert the irinotecan (open-lactone ring, inactive form) to the active form of the drug and allow its migration into the lipid bilayer.

### EXAMPLE 4

Lipids were dissolved in ethanol. The lipid alcohol mixture was then dispersed in irinotecan/sucrose solution pH at 8-10. The bulk liposomal irinotecan was then extruded through 0.2 μM and 0.1 μM polycarbonate filters. Following size-reduction, the product was then heated under vacuum to evaporate the organic solvent and then sterile filtered through 0.22 μM filters and lyophilized.

### EXAMPLE 5

Irinotecan liposome complexes were prepared using the following procedure. The lipids were mixed with cardiolipin. The mixed powdered lipids were dissolved in chloroform in a round bottomed flask. The clear solution was placed on a Buchi rotary evaporator to make a thin film. The flask containing the thin lipid film was dried under vacuum. The film was hydrated in an irinotecan alkaline solution containing sucrose. The hydrated lipid film was rotated. The mixture in the flask was vortexed and mixed. The mixture was sequentially extruded through decreasing size filters of 800 nm, 400 nm, 200 nm, and 100 nm. The irinotecan liposome complexes were then lyophilized under vacuum. The resulting dehydrated complexes can be stored at 2-8° C. for at least 12 months. Prior to administration, the irinotecan can be activated by adding acidic buffer.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of forming a lipid composition comprising a compound selected from a group consisting of irinotecan and a chemical in equilibrium with irinotecan, involving forming a lipid phase and thereafter hydrating the lipid phase with a first aqueous solution including the compound so as to form a lipid composition including the compound, and thereafter reducing the pH of the lipid composition including the compound to a pH of less than about 3.5.

2. The method of claim 1, wherein the lipid phase is formed in an organic solvent.

3. The method of claim 1, wherein the first aqueous solution has an alkaline pH.

4. The method of claim 3, wherein the pH of the first aqueous solution is between about 7 and about 11.

5. The method of claim 3, wherein the pH of the first aqueous solution is between about 8 and about 10.

6. The method of claim 1, wherein the hydration step is performed with vigorous mixing.

7. The method of claim 1, wherein the pH of the lipid composition including the compound is reduced to between about 1.0 and about 3.5.

8. The method of claim 1, wherein the pH of the lipid composition including the compound is reduced to between about 1.5 and about 3.0.

9. The method of claim 1, wherein the pH of the lipid composition including the compound is reduced to about 2.7.

10. The method of claim 1, further involving filtering the lipid composition.

11. The method of claim 10, wherein the filtration occurs prior to reducing the pH of the composition.

12. The method of claim 1, further involving dehydrating the lipid composition to form a dried lipid composition.

13. The method of claim 12, wherein the drying occurs prior to reducing the pH of the composition.

14. The method of claim 1, further involving adding a protective sugar to the lipid composition.

15. The method of claim 1, wherein the reduction of pH is achieved with the addition of an acidic polar solvent.

16. The method of claim 15, wherein the polar solvent is an aqueous solution with an acidic pH less than about 3.5.

17. The method of claim 15, wherein the polar solvent is an aqueous solution with an acidic pH between about 1.0 and about 3.5.

18. The method of claim 15, wherein the polar solvent is an aqueous solution with an acidic pH between about 1.5 and about 3.0.

19. The method of claim 15, wherein the polar solvent is an aqueous solution with an acidic pH less than about 2.0.

20. The method of claim 1, wherein the lipid phase comprises cardiolipin.

21. The method of claim 20, wherein the cardiolipin is selected from a group consisting of natural cardiolipin, synthetic cardiolipin, and chemically-modified cardiolipin.

22. The method of claim 20, wherein the lipid phase further comprises at least one of the lipids selected from a group of lipids consisting of phosphatidylcholine, phosphatidylethanolamine, phosphatidylserine, phosphatidylglycerol, phosphatidic acid, phosphatidylinositol, sphingomyelin, sterol, tocopherol, fatty acid, and mixtures thereof.

23. The method of claim 22, wherein the phosphatidylglycerol is selected from a group consisting of dimyristoylphosphatidylglycerol, dioleoylphosphatidylglycerol, distearoylphosphatidylglycerol, dipalmitoylphosphatidylglycerol, diarachidonoylphosphatidylglycerol and mixtures thereof.

24. The method of claim 22, wherein the phosphatidylcholine is selected from a group consisting of dimyristoylphosphatidylcholine, distearoylphosphatidyl choline, dioleoylphosphatidylcholine, dipalmitoylphosphatidylcholine, diarachidonoyl phosphatidylcholine, egg phosphatidylcholine, soy phosphatidylcholine, hydrogenated soy phosphatidylcholine, and mixtures thereof.

25. The method of claim 22, wherein the sterol is selected from a group consisting of cholesterol, polyethylene glycol derivatives of cholesterol, coprostanol, cholestanol, cholestane, cholesterol hemisuccinate, cholesterol sulfate, and mixtures thereof.
